(19) United States
(12) Patent Application Publication
(10) Pub. No.: US 2013/0216429 A1
(43) Pub. Date: Aug. 22, 2013

(54) Method for disinfecting bedding items by microwaves, in particular mattresses, and related facility

(71) Applicants: Rémy Lanza, Bollene (FR); Salim Touati, Livron Sur Drome (FR)

(72) Inventors: Rémy Lanza, Bollene (FR); Salim Touati, Livron Sur Drome (FR)

(73) Assignee: ECOVAL ENVIRONNEMENT, Lognes (FR)

(21) Appl. No.: 13/812,996

(22) PCT Filed: Jul. 28, 2011
(86) PCT No.: PCT/FR2011/051821

§ 371 Date: Apr. 5, 2013

(30) Foreign Application Priority Data
Jul. 29, 2010 (FR) 10 56251

(51) Int. Cl. A61L 2/12; H05B 1/02

(52) U.S. Cl. CPC A61L 2/12; H05B 1/02
USPC 422/21; 219/702

(57) ABSTRACT

The invention relates to a facility for disinfecting bedding items, in particular mattresses or cushions, the facility (30) including:
a device (38) for generating microwaves which is capable of being selectively activated in order to expose the bedding item to microwave radiation, the power of which is 40 kW to 100 kW,
a member (66) for controlling the microwave-generating device (38), programmed to activate the microwave-generating device (38) for an exposure time suitable for maintaining the central layer of the bedding item at a temperature of more than 70° C. for a predetermined period of time that is longer than 45 seconds.

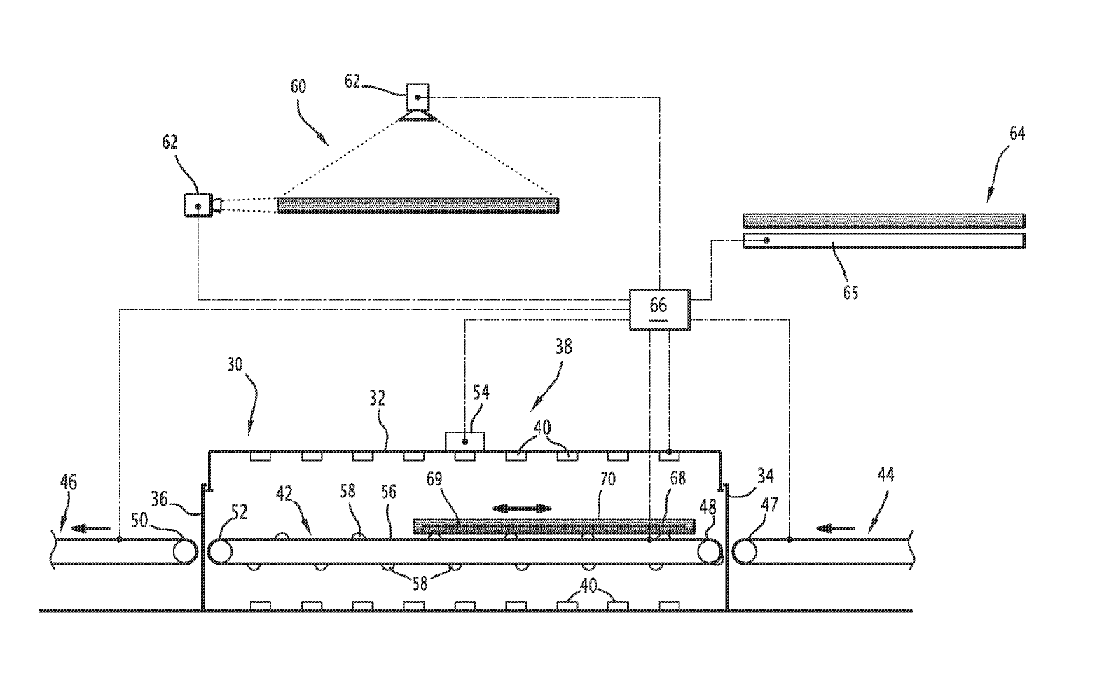

The present invention generally relates to the field of bedding.

More specifically, according to a first aspect, the invention relates to a method for disinfecting bedding items, in particular mattresses or cushions.

Mattresses may be recycled at the end of their life. In such a recycling operation, certain tasks must be performed manually by operators. In order to protect the operators and guarantee complete hygiene of the recycled products, the mattresses must first be disinfected.

Known from EP24304 is a sterilization method that combines the application of disinfectant solutions and microwave heating. Such a method is complex to carry out.

In this context, the invention aims to propose a method for disinfecting bedding items, in particular mattresses or cushions, that is easier to use.

To that end, the invention relates to a method for disinfecting bedding items, in particular mattresses, the method comprising a step for exposing the bedding item to microwave radiation, the power of which is 40 kW to 100 kW, for an exposure time suitable for maintaining a central layer of the bedding item at a temperature of more than 70° C. for at least 45 seconds.

The method may also have one or more of the features below, considered individually or according to all technically possible combinations:
the bedding item is exposed to microwave radiation with a power of between 8 and 25 kW per square meter of surface area of the large surfaces of the bedding item;
the total exposure time is broken down into a heating time suitable for making the central layer of the bedding element go from an initial temperature of less than 70° C. to a final temperature of more than 70° C., plus the predetermined period of time;
the bedding item has a thickness comprised between 5 and 25 cm and two large surfaces each having a given surface area, the heating duration expressed in seconds being comprised between the following lower and upper bounds:

Lower bound BI=15×S

Upper bound BS=35×S

where S is the given surface area of one of the large surfaces of the bedding item expressed in square meters;
the bedding item has a thickness comprised between 25 and 40 cm and two large surfaces each having a given surface area, the heating duration expressed in seconds being comprised between the following lower and upper bounds:

Lower bound BI=35×S

Upper bound BS=70×S

where S is the given surface area of one of the large surfaces of the bedding item expressed in square meters;
the bedding item is a mattress having a core made from a foam primarily comprising polyurethane, the mattress having a thickness comprised between 5 and 15 cm and two large surfaces each having a given surface area, the heating duration expressed in seconds being comprised between the following lower and upper bounds:

Lower bound BI=10×S

Upper bound BS=25×S

where S is the given surface area of one of the large surfaces of the bedding item expressed in square meters;
the bedding item is a mattress having a foam core primarily comprising polyurethane, the mattress having a thickness comprised between 15 and 25 cm and two large surfaces each having a given surface area, the heating duration expressed in seconds being comprised between the following lower and upper bounds:

Lower bound BI=15×S

Upper bound BS=30×S

where S is the given surface area of one of the large surfaces of the bedding item expressed in square meters;
the bedding item is a mattress having a foam core primarily comprising polyurethane, the mattress having a thickness comprised between 25 and 35 cm and two large surfaces each having a given surface area, the heating duration expressed in seconds being comprised between the following lower and upper bounds:

Lower bound BI=30×S

Upper bound BS=60×S

where S is the given surface area of one of the large surfaces of the bedding item expressed in square meters;
the bedding item is a mattress having a foam core primarily comprising latex, the mattress having a thickness comprised between 5 and 15 cm and two large surfaces each having a given surface area, the heating duration expressed in seconds being comprised between the following lower and upper bounds:

Lower bound BI=20×S

Upper bound BS=40×S

where S is the given surface area of one of the large surfaces of the bedding item expressed in square meters; and
the bedding element is a mattress having a foam core primarily comprising latex, the mattress having a thickness comprised between 15 and 25 cm and two large surfaces each having a given surface area, the heating duration expressed in seconds being comprised between the following lower and upper bounds:

Lower bound BI=30×S

Upper bound BS=60×S

where S is the given surface area of one of the large surfaces of the bedding item expressed in square meters;
the bedding element is a mattress having a foam core primarily comprising latex, the mattress having a thickness comprised between 25 and 35 cm and two large surfaces each having a given surface area, the heating duration expressed in seconds being comprised between the following lower and upper bounds:

Lower bound BI=40×S

Upper bound BS=80×S

where S is the given surface area of one of the large surfaces of the bedding item expressed in square meters;
the bedding element is moved during the step for exposure to the microwave radiation;
it comprises a step for acquiring a parameter representative of the weight of the bedding element preceding the exposure step, the exposure duration and the power of the microwave radiation being chosen at least as a function of the acquired parameter;
it comprises a step for acquiring one or more parameters representative of one or more dimensions of the bedding element preceding the exposure step, the exposure duration and the power of the microwave radiation being chosen at least as a function of the acquired parameter(s).

According to a second aspect, the invention relates to a facility for disinfecting bedding items, in particular mattresses or cushions, the facility comprising:
a microwave generator capable of being activated selectively to expose the bedding item to microwave radiation with a power comprised between 40 kW and 100 kW,
a control member for the microwave generator, programmed to activate the microwave generator for an exposure time suitable for maintaining a central layer of the bedding item at a temperature of more than 70° C. for at least 45 seconds.

Other features and advantages of the invention will emerge from the detailed description thereof provided below, for information and non-limitingly, in reference to the sole appended figure, diagrammatically showing the microwave disinfection facility.

The method that will be described below is intended to disinfect bedding items at the end of their life, for example before dismemberment and recycling of those bedding items. Said bedding items are typically preferably mattresses or cushions, but are not limited thereto.

The items to be processed are typically:
foam mattresses, which comprise a textile enclosure and a foam core housed inside the textile enclosure, the foam including a majority of polyurethane; or
latex mattresses, which include a textile enclosure, and a latex core housed inside the textile enclosure.

These mattresses are generally parallelepiped and typically, but not exclusively, have one of the following sizes:
length 190 cm and width 90 cm;
length 190 cm and width 140 cm;
length 200 cm and with 160 cm;
length 200 cm and with 180 cm.

The mattresses typically, but not exclusively, have one of the following thicknesses: 10 cm, 20 cm or 30 cm.

The purpose of the disinfection operation is to destroy the bacteriological germs that may be present in the items to be treated. The disinfection must be sufficient from a sanitary perspective to protect the operators performing the various operations necessary to recycle the mattresses (dismemberment of the mattresses, sorting of the materials, etc.), and to guarantee complete hygiene of the finished product obtained from the recycled materials.

The disinfection operation is not a sterilization operation and does not aim to destroy all of the germs present in the items to be processed.

The disinfection operation aims to eliminate at least 99% of the bacteriological germs, preferably at least 99.9% of the bacteriological germs, still more preferably at least 99.99% of the bacteriological germs.

The disinfecting method comprises a step for exposing the bedding item to microwave radiation with a power comprised between 40 kW and 100 kW, for an exposure time that is suitable to maintain a central layer of the bedding item at a temperature of more than 70° C. for a predetermined period of time.

The central layer of the mattress corresponds to the core of the mattress. This is the layer that is furthest from the outer surface of the mattress. It for example has a thickness of three centimeters perpendicular to the large surfaces of the mattress. It extends, in a plane parallel to the two large surfaces, for example up to approximately 3 cm from the small surfaces of the mattress, i.e., the four surfaces making up the sides of the mattress. The large surfaces are the two opposite surfaces having the largest sizes.

The temperature of 70° C. is sufficient for disinfection, but not for sterilization. The exposure time is broken down into:
a heating duration suitable for causing the central layer of the bedding item to go from an initial temperature of less than 70° C. to a final temperature of more than 70° C.,
plus the predetermined period of time during which the central layer is maintained at the final temperature.

The predetermined duration is equal to at least 45 seconds, preferably at least 60 seconds, and is typically comprised between 60 seconds and 90 seconds.

The initial temperature corresponds to the ambient temperature, and is generally close to 20° C. The heating duration is therefore chosen to cause a temperature increase of the central layer of at least 50° C., for example 60° C.

The final temperature is comprised between 70° C. and 90° C., and preferably between 70° C. and 80° C.

The microwave radiation power depends on the dimensions of the mattress to be treated (length, width, thickness) and the material making up the core of the mattress (polyurethane foam or latex).

Typically, the bedding item is exposed to microwave radiation having a power comprised between 8 and 25 kW per square meter of total surface area of the large surfaces of the bedding element (sum of the surface areas of the two large surfaces.

For a core made from polyurethane foam, the power is comprised between 60 kW XX1 and 80 kW XX2 per square meter of the total surface area of the large surfaces. Irrespective of the surface to be treated and the definition of the material, the same power of approximately 60 kW or 80 kW is applied, and only the exposure time at one of those two powers changes. For a latex core, the power is 60 kW and 80 kW per square meter of total surface area of the large surfaces.

The heating duration also depends on the dimensions of the mattress to be treated (length, width, thickness) and the material making up the core of the mattress (polyurethane foam or latex).

Upon first approximation, for a mattress with a thickness comprised between 5 and 25 cm, the heating duration expressed in seconds is comprised between the following lower and upper bounds:

Lower bound BI=15×S

Upper bound BS=35×S

where S is the given surface area of one of the large surfaces of the bedding item expressed in square meters.

For a mattress having a thickness comprised between 25 and 40 cm, the heating duration expressed in seconds is comprised between the following lower and upper bounds:

Lower bound BI=35×S

Upper bound BS=70×S

where S is the given surface area of one of the large surfaces of the bedding item expressed in square meters.

More specifically, if the bedding item is a mattress having a core made from a foam primarily comprising polyurethane, the mattress having a thickness comprised between 5 and 15 cm, the heating duration expressed in seconds is comprised between the following lower and upper bounds:

Lower bound BI=10×S

Upper bound BS=25×S

where S is the given surface area of one of the large surfaces of the bedding item expressed in square meters. Preferably, said heating duration is comprised between 15×S and 22×S.

If the bedding item is a mattress having a foam core primarily comprising polyurethane, the mattress having a thickness comprised between 15 and 25 cm, the heating duration expressed in seconds is comprised between the following lower and upper bounds:

Lower bound BI=15×S

Upper bound BS=30×S

where S is the given surface area of one of the large surfaces of the bedding item expressed in square meters. Preferably, said heating duration is comprised between 20×S and 28×S.

If the bedding element is a mattress with a foam core primarily comprising polyurethane, the mattress having a thickness comprised between 25 and 35 cm, the heating duration expressed in seconds is comprised between the following lower and upper bounds:

Lower bound BI=30 ×S

Upper bound BS=60 ×S

where S is the given surface area of one of the large surfaces of the bedding item expressed in square meters. Preferably, said heating duration is comprised between 39×S and 53×S.

If the bedding item is a mattress having a foam core primarily comprising latex, the mattress having a thickness comprised between 5 and 15 cm, the heating duration expressed in seconds is comprised between the following lower and upper bounds:

Lower bound BI=20×S

Upper bound BS=40×S

where S is the given surface area of one of the large surfaces of the bedding item expressed in square meters. Preferably, said heating duration is comprised between 20×S and 33×S.

If the bedding item is a mattress with a foam core primarily comprising latex, the mattress having a thickness comprised between 15 and 25 cm, the heating duration expressed in seconds is comprised between the following lower and upper bounds:

Lower bound BI=30×S

Upper bound BS=60×S

where S is the given surface area of one of the large surfaces of the bedding item expressed in square meters. Preferably, said heating duration is comprised between 25×S and 50×S.

If the bedding item is a mattress having a foam core primarily comprising latex, the mattress having a thickness comprised between 25 and 35 cm, the heating duration expressed in seconds is comprised between the following lower and upper bounds:

Lower bound BI=40×S

Upper bound BS=80×S

where S is the given surface area of one of the large surfaces of the bedding item expressed in square meters. Preferably, said heating duration is comprised between 45×S and 65×S.

Tests were carried out to determine the heating duration for various mattresses. The results are shown in the following tables.

Foam

| | Mattress Size | | Heating time (s) | | |
|---|---|---|---|---|---|
| | Length × | Surface | Thick- | Thick- | Thick- |
| Microwave | Width | area | ness | ness | ness |
| Power (kW) | (cm) | (m²) | 10 cm | 20 cm | 30 cm |
| 80 | 90 × 190 | 1.71 | 30 | 41 | 75 |
| 80 | 140 × 190 | 2.66 | 45 | 52 | 110 |
| 80 | 180 × 200 | 3.6 | 56 | 71 | 135 |
| 60 | 90 × 190 | 1.71 | 40 | 55 | 100 |
| 60 | 140 × 190 | 2.66 | 60 | 70 | 147 |
| 60 | 180 × 200 | 3.6 | 75 | 95 | 180 |

Latex

| | Mattress Size | | Heating time (s) | | |
|---|---|---|---|---|---|
| | Length × | Surface | Thick- | Thick- | Thick- |
| Microwave | Width | area | ness | ness | ness |
| Power (kW) | (cm) | (m²) | 10 cm | 20 cm | 30 cm |
| 80 | 90 × 190 | 1.71 | 33 | | 83 |
| 80 | 140 × 190 | 2.66 | 65 | | 128 |
| 80 | 180 × 200 | 3.6 | 90 | | 175 |
| 60 | 90 × 190 | 1.71 | 45 | | 110 |
| 60 | 140 × 190 | 2.66 | 90 | | 170 |
| 60 | 180 × 200 | 3.6 | 120 | | 230 |

The mattress is preferably moved during the step for exposure to the microwave radiation, so as to obtain uniform microwave exposure of the different areas of the mattress to the microwaves and avoid hotspots. Foam mattresses may be treated statically, without moving the mattress during the microwave exposure. Latex mattresses are preferably moved during treatment.

The choice of the microwave radiation power and the exposure time is preferably made automatically by a computer. Alternatively, an operator chooses the power and/or the exposure time.

To that end, the computer is provided with the following information for each mattress: thickness, size (length, width), material of the core (latex, polyurethane foam).

The size and/or thickness may be measured automatically by sensors provided to that end, the results of the measurements being communicated to the computer automatically. The sensors are for example image acquisition devices, associated with an image analysis device. The size and/or thickness may also be entered manually into the memory of the computer by an operator, who estimates them or measures them directly.

The component material of the core may be determined in various ways.

An automatic cut-through resistance test may be done (carrying capacity measurement), and the measured value provided to the computer automatically. The computer automatically determines the material as a function of said value, using a table or a graph. In fact, a cut-through resistance value range may be associated with each of the two materials.

Alternatively, the cut-through resistance can be determined manually by an operator. The operator deduces the material making up the core therefrom, and provides information to the computer.

The disinfecting facility comprises a treatment tunnel 30, shown in the FIGURE.

The treatment tunnel comprises:
an enclosure 32, for example in the form of a tunnel, provided with an entry door 34 at a first end, and an outlet door 36 at a second end opposite the first;
a microwave generating device 38, equipped with a plurality of microwave generating elements 40 placed in the tunnel 32;
a primary conveyor 42, arranged inside the tunnel 32 and provided to move the elements to be treated between the two ends of the tunnel 32;
a conveyor 44 bringing in the items to be treated;
a conveyor 46 discharging the items to be treated after passage in the tunnel.

The microwave generating device 38 is suitable for generating microwave radiation with an adjustable power. The maximum power of the microwave radiation generated by the device 38 is 80 kW. Alternatively, the maximum power is 60 kW.

The frequency of the magnetron that generates the microwaves is approximately 2450 MHz.

The microwave generating elements 40 are distributed above and below the conveyor 42. They are distributed over the entire length of the tunnel 32, from the first end to the second end of that tunnel. Each element 40 can generate a microwave radiation with an adjustable unitary power, the maximum power being comprised between 2 and 3 kW.

The doors 34 and 36 are for example of the guillotine type.

The entry door 44 is arranged such that its end 47 adjoins one end 48 of the primary conveyor 42, the entry door 34 being situated between the ends 47 and 48. Symmetrically, the discharge conveyor 46 is placed such that its end 50 adjoins the end 52 of the primary conveyor, the end 52 being opposite the end 48. The door 36 is situated between the ends 50 and 52.

Furthermore, the tunnel includes a probe 54 for measuring the surface temperatures of the item to be treated. The probe 54 operates by infrared pyrometry, and is suitable for measuring the surface temperatures during the microwave treatment.

The conveyor 42 is for example a belt conveyor. It extends over the entire length of the tunnel 42. The belt has an outer surface area 56 and raised portions 58 protruding relative to the surface area 56. The raised portions 58 are small and are distributed over the entire outer surface area of the belt. The mattress 57 undergoing treatment rests on the raised portions 58. In this way, the large surface of the mattress facing the belt is not in contact with the visible surface area 56. It is only in contact with the raised portions 58, and over a very small fraction of its surface area (typically less than 5% of its surface). The rest of the surface area of the large surface is free.

The facility may include a station 60 for measuring the size (length/width) and carrying capacity of the mattress. This device for example includes one or more cameras 62, associated with image analysis means.

The facility also includes a computer 66, provided to drive the microwave generating device 38, the conveyors 42, 44 and 46, the doors 34 and 36, and the temperature probe 54. It receives the results of the measurements done by the measuring devices 60 and 64. The computer is programmed to determine the power of the microwave radiation and the length of treatment for each item to be treated, as a function of the results of measurements received from the devices 60 and 64, or as a function of data input by an operator if necessary.

The operation of the tunnel is as follows.

After having gone through the stations for measuring the weight 64 and the size/thickness 60, the items to be treated are brought one after the other by the intake conveyor 44 up to the entry door 34. They are treated one by one inside the tunnel 32.

The computer 66 commands the opening of the entry door 34. It then commands that the conveyors 42 and 44 be started, so as to cause the item to be treated 68 to penetrate the inside of the housing 32. The computer 66 then commands closing of the entry door 34, then operation of the microwave generating device 38. The power of the microwaves emitted by the emitters 40 and the exposure time of the items 68 to the microwaves are commanded by the computer 66 according to the characteristics of the item to be treated.

These parameters make it possible to bring the central layer 69 of the item to be treated to a temperature of more than 70° C. and to maintain that layer at said temperature for a period of time suitable for performing the disinfection. The surface layer 70, which surrounds the layer 69, is at a higher temperature than the layer 69 and is also disinfected.

During emission of the microwaves, the element 68 is moved inside the tunnel by the primary conveyor 42 following to-and-fro movements between the two ends of the tunnel 32.

Once the microwave treatment is completed, the computer commands the opening of the outlet door 36, then starts the conveyors 42 and 46, so as to transfer the treated item from the conveyor 42 to the conveyor 46.

The computer then commands closure of the door 36 and opening of the door 34, so as to allow another item to be loaded inside the tunnel 32.

The fact that the method comprises a step for exposing the bedding item to microwave radiation having a power comprised between 40 kW and 100 kW, for a total exposure time suitable for maintaining the central layer of the bedding item at a temperature of more than 70° C. for a predetermined period of time greater than 45 seconds, makes it possible to disinfect the bedding items simply and effectively. The method is fast, and does not require the use of chemical disinfectant solutions.

Surprisingly, the applicant discovered that the heating times may be expressed simply, as a function of the surface area of the large surfaces of the bedding items, which allows easy automatic treatment of the bedding items.

The automation may be even more developed when the method comprises a step for acquiring the carrying capacity of the bedding item and/or the dimensions of the bedding item (thickness, size), then making it possible to choose the power and the duration of the heating phase automatically.

1. A method for disinfecting bedding items, in particular mattresses, the method comprising a step for exposing the bedding item to microwave radiation, the power of which is 40 kW to 100 kW, for an exposure time suitable for maintaining a central layer of the bedding item at a temperature of more than 70° C. for at least 45 seconds.

2. The method according to claim 1, characterized in that the bedding item is exposed to microwave radiation with a power of between 8 and 25 kW per square meter of surface area of the large surfaces of the bedding item.

3. The method according to claim 1, characterized in that the total exposure time is broken down into a heating time suitable for making the central layer of the bedding element go from an initial temperature of less than 70° C. to a final temperature of more than 70° C., plus the predetermined period of time.

4. The method according to claim 3, characterized in that the bedding item has a thickness comprised between 5 and 25 cm and two large surfaces each having a given surface area, the heating duration expressed in seconds being comprised between the following lower and upper bounds:
Lower bound BI=15×S
Upper bound BS=35×S
where S is the given surface area of one of the large surfaces of the bedding item expressed in square meters.

5. The method according to claim 3, characterized in that the bedding item has a thickness comprised between 25 and 40 cm and two large surfaces each having a given surface area, the heating duration expressed in seconds being comprised between the following lower and upper bounds:
Lower bound BI=35×S
Upper bound BS=70×S
where S is the given surface area of one of the large surfaces of the bedding item expressed in square meters.

6. The method according to claim 3, characterized in that the bedding item is a mattress having a core made from a foam primarily comprising polyurethane, the mattress having a thickness comprised between 5 and 15 cm and two large surfaces each having a given surface area, the heating duration expressed in seconds being comprised between the following lower and upper bounds:
Lower bound BI=10×S
Upper bound BS=25×S
where S is the given surface area of one of the large surfaces of the bedding item expressed in square meters.

7. The method according to claim 3, characterized in that the bedding item is a mattress having a foam core primarily comprising polyurethane, the mattress having a thickness comprised between 15 and 25 cm and two large surfaces each having a given surface area, the heating duration expressed in seconds being comprised between the following lower and upper bounds:
Lower bound BI=15×S
Upper bound BS=30×S
where S is the given surface area of one of the large surfaces of the bedding item expressed in square meters.

8. The method according to claim 3, characterized in that the bedding item is a mattress having a foam core primarily comprising polyurethane, the mattress having a thickness comprised between 25 and 35 cm and two large surfaces each having a given surface area, the heating duration expressed in seconds being comprised between the following lower and upper bounds:
Lower bound BI=30×S
Upper bound BS=60×S
where S is the given surface area of one of the large surfaces of the bedding item expressed in square meters.

9. The method according to claim 3, characterized in that the bedding item is a mattress having a foam core primarily comprising latex, the mattress having a thickness comprised between 5 and 15 cm and two large surfaces each having a given surface area, the heating duration expressed in seconds being comprised between the following lower and upper bounds:
Lower bound BI=20×S
Upper bound BS=40×S
where S is the given surface area of one of the large surfaces of the bedding item expressed in square meters.

10. The method according to claim 3, characterized in that the bedding element is a mattress having a foam core primarily comprising latex, the mattress having a thickness comprised between 15 and 25 cm and two large surfaces each having a given surface area, the heating duration expressed in seconds being comprised between the following lower and upper bounds:
Lower bound BI=30×S
Upper bound BS=60×S
where S is the given surface area of one of the large surfaces of the bedding item expressed in square meters.

11. The method according to claim 3, characterized in that the bedding element is a mattress having a foam core primarily comprising latex, the mattress having a thickness comprised between 25 and 35 cm and two large surfaces each having a given surface area, the heating duration expressed in seconds being comprised between the following lower and upper bounds:
Lower bound BI=40×S
Upper bound BS=80×S
where S is the given surface area of one of the large surfaces of the bedding item expressed in square meters.

12. The method according to claim 1, characterized in that the bedding element is moved during the step for exposure to the microwave radiation.

13. The method according to claim 1, characterized in that it comprises a step for acquiring a parameter representative of the weight of the bedding element preceding the exposure step, the exposure duration and the power of the microwave radiation being chosen at least as a function of the acquired parameter.

14. The method according to claim 1, characterized in that it comprises a step for acquiring one or more parameters representative of one or more dimensions of the bedding element preceding the exposure step, the exposure duration and the power of the microwave radiation being chosen at least as a function of the acquired parameter(s).

15. A facility for disinfecting bedding items, in particular mattresses or cushions, the facility (30) comprising:
a microwave generator device (38) capable of being activated selectively to expose the bedding item to microwave radiation with a power comprised between 40 kW and 100 kW,
a control member (66) for the microwave generator (38), programmed to activate the microwave generator (38) for an exposure time suitable for maintaining a central layer of the bedding item at a temperature of more than 70° C. for at least 45 seconds.
